(19) United States
(12) Patent Application Publication
(10) Pub. No.: US 2025/0325053 A1
(43) Pub. Date: Oct. 23, 2025

(54) EYE PROTECTION APPARATUS

(71) Applicant: Joseph Bryan Guzzardi, Coos Bay, OR (US)

(72) Inventor: Joseph Bryan Guzzardi, Coos Bay, OR (US)

(21) Appl. No.: 19/074,695

(22) Filed: Mar. 10, 2025

Related U.S. Application Data
(60) Provisional application No. 63/635,637, filed on Apr. 18, 2024.

(51) Int. Cl. A41D 13/11; A61F 9/02

(52) U.S. Cl. CPC A41D 13/1184; A61F 9/025

(57) ABSTRACT

An eye protection apparatus comprising: a headgear, comprising a head covering and a protruding portion with a slit, attached to an outer portion of the head covering, an eyeshield having an eye-shielding surface, and a positioning mechanism comprising at least one attachment portion for attaching the positioning mechanism to the head covering of the headgear, at least one securing apparatus that secures the eyeshield to the positioning mechanism, and at least one movement apparatus configured to allow movement of the at least one securing apparatus and the eyeshield secured to the at least one securing apparatus through the slit of the protruding portion of the headgear, wherein the positioning mechanism is configured to alleviate pressure of the eyeshield on a user's face and head.

CONTINUITY AND CLAIM OF PRIORITY

This is a U.S. non-provisional patent application claiming the benefit and priority of a co-pending U.S. provisional Patent Application Ser. No. 63/635,637, filed on Apr. 18, 2024.

### BACKGROUND OF THE INVENTION

### Technical Field

The invention relates generally to an eyeshield, such as sunglasses, safety glasses, eye visors, and prescription glasses, and, more particularly, the invention relates to an eyeshield on a positioning mechanism attached or attachable to a headgear, such as a hat or a cap, that is configured to alleviate pressure of the eyeshield on a user's face and head.

### Description of Related Art

Eyeshields that protect the eye from various hazards, such as, but not limited to, impact, debris, chemicals, water, light, or UV-rays such as sun glasses, blue light glasses, safety glasses, swim googles, and eye visors are well known in the art. Eyeshields with additional functionalities, such as, but not limited to, prescription glasses, 3D glasses, augmented reality glasses, and even virtual reality headsets, are well known in the art.

Various headgears, such as, but not limited to, hats, caps, beanies, berets, helmets, and cowboy hats are known in the art. Some headgears with attached eyeshields, such as protective face shields, and helmets for activities such as snowboarding and motorcycling, are well known in the art.

### BRIEF SUMMARY OF THE INVENTION

The present invention is an eye protection apparatus comprising a headgear, an eyeshield, and a positioning mechanism. The headgear comprises a head covering and a protruding portion with a slit. The protruding portion is attached to an outer portion of the head covering. The eyeshield has an eye-shielding surface. The positioning mechanism comprises at least one attachment portion, at least one securing apparatus, and at least one movement apparatus. The attachment portion is for attaching the positioning mechanism to the head covering of the headgear. The securing apparatus secures the eyeshield to the positioning mechanism. The movement apparatus is configured to allow movement of the securing apparatus and the eyeshield secured to the securing apparatus through the slit of the protruding portion of the headgear. The movement apparatus is also configured to allow the securing apparatus and the eyeshield secured to the securing apparatus to tilt at an angle when the eyeshield is positioned below the slit. The positioning mechanism is configured to alleviate pressure of the eyeshield on a user's face and head.

In a preferred embodiment, the headgear is a cap and the protruding portion is a visor of the cap.

In a preferred embodiment, the movement apparatus is comprised of a guide rail and the securing apparatus is configured to move along the guide rail of the movement apparatus.

In an embodiment, the positioning mechanism is configured to allow a user to manually control the movement of the securing apparatus and the eyeshield secured to the securing apparatus through the slit of the protruding portion of the headgear.

In another embodiment, the positioning mechanism is configured to allow a user to mechanically control the movement of the securing apparatus and the eyeshield secured to the securing apparatus through the slit of the protruding portion of the headgear.

In yet another embodiment, the positioning mechanism is configured to allow a user to electromechanically control the movement of the securing apparatus and the eyeshield secured to the securing apparatus through the slit of the protruding portion of the headgear. The positioning mechanism can further comprise a button configured to allow the user to electromechanically control the movement of the securing apparatus and the eyeshield secured to the securing apparatus through the slit of the protruding portion of the headgear.

In an embodiment, the eyeshield embodies one or more properties, which can be UV protection, radiation protection, visible light filtration, impact resistance, durability, polarization, optometric prescription, ANSI Z87.1, 3D binocular vision, multifocal, prism correction, high refraction index, scratch resistant, anti-reflective, water-repellant, light-responsive, and flash mirrored. The eye-shielding surface of the eyeshield can be made of glass, plastic, polycarbonate, trivex, high-index plastic, acetate, CR39 plastic, or polyacrylate, or any other material known in the art. A position of the eyeshield can be locked by a locking mechanism of the positioning mechanism, such as detents, springs, or magnetic material, or any other locking mechanism known in the art.

In an embodiment, the headgear and the positioning mechanism can be comprised of one or more of fiber, fabric, metal polymer, and composite materials, and any other materials and mixture of materials known in the art. The headgear and the positioning mechanism can be made of multiple portions, with each portion using different materials.

In an embodiment, the eye protection apparatus may be just comprised of an eyeshield having an eye-shielding surface and a positioning mechanism, the positioning mechanism comprising at least one attachment portion, at least one securing apparatus, and at least one movement apparatus. The securing apparatus secures the eyeshield to the positioning mechanism. The attachment portion of the positioning mechanism can be used to attach the eye protection apparatus to a separately manufactured or provided headgear. The movement apparatus is configured to allow movement of the securing apparatus and the eyeshield secured to the securing apparatus through a slit in a protruding portion of the headgear.

### BRIEF DESCRIPTIONS OF THE DRAWINGS

FIG. 1A is a side perspective view of an embodiment of the eye protection apparatus having a headgear having a head covering and a protruding portion with a slit, an eyeshield, and a positioning mechanism having two attachment portions, two securing apparatuses, and two movement apparatuses, with the eyeshield positioned below the slit. A handle is provided for manual control of the positioning mechanism.

FIG. 1B is a side perspective view of the embodiment of the eye protection apparatus shown in FIG. 1A, with the eyeshield positioned above the slit.

FIG. 1C is the side perspective view of the embodiment of the eye protection apparatus shown in FIG. 1B, with a button provided for mechanical control of the positioning mechanism.

FIG. 1D is the side perspective view of the embodiment of the eye protection apparatus shown in FIGS. 1B-1C, with a button provided for electromechanical control of the positioning mechanism.

FIG. 2A is a front view of another embodiment of the eye protection apparatus having a headgear having a head covering and a protruding portion with a slit, an eyeshield, and a positioning mechanism having two attachment portions, two securing apparatuses, and two movement apparatuses, with the eyeshield positioned above the slit.

FIG. 2B is a front view of the embodiment of the eye protection apparatus shown in FIG. 2A, with the eyeshield positioned below the slit.

FIG. 3A is a left side view of the embodiment of the eye protection apparatus shown in FIGS. 2A-2B, with the eyeshield positioned above the slit.

FIG. 3B is a left side view of the embodiment of the eye protection apparatus shown in FIGS. 2A-3A, with the eyeshield positioned below the slit.

FIG. 4A is a back view of the embodiment of the eye protection apparatus shown in FIGS. 2A-3B, with the eyeshield positioned above the slit.

FIG. 4B is a back view of the embodiment of the eye protection apparatus shown in FIGS. 2A-4A, with the eyeshield positioned below the slit.

FIG. 5A is a right side view of the embodiment of the eye protection apparatus shown in FIGS. 2A-4B, with the eyeshield positioned above the slit.

FIG. 5B is a right side view of the embodiment of the eye protection apparatus shown in FIGS. 2A-5A, with the eyeshield positioned below the slit.

FIG. 6A is a top view of the embodiment of the eye protection apparatus shown in FIGS. 2A-5B.

FIG. 6B is a bottom view of the embodiment of the eye protection apparatus shown in FIGS. 2A-6A.

FIG. 7A is a side view of another embodiment of the eye protection apparatus having a headgear having a head covering and a protruding portion with a slit, an eyeshield, and a positioning mechanism having two attachment portions, two securing apparatuses, and two movement apparatuses, showing that the movement apparatus is configured to allow the securing apparatus and the attached eyeshield to tilt when the eyeshield is positioned below the slit.

FIG. 7B is a side view of the eye protection apparatus shown in FIG. 7A, also showing that the movement apparatus is configured to allow the securing apparatus and the attached eyeshield to tilt when the eyeshield is positioned below the slit.

FIG. 8A is a front view of an embodiment of the eyeshield and a positioning mechanism of the eye protection apparatus.

FIG. 8B is a back view of the eyeshield and the positioning mechanism of the eye protection apparatus shown in FIG. 8A.

FIG. 8C is a side view of the eyeshield and the positioning mechanism of the eye protection apparatus shown in FIGS. 8A-8B.

FIG. 8D is a top view of the eyeshield and the positioning mechanism of the eye protection apparatus shown in FIGS. 8A-8C.

FIG. 9A is a side above perspective view of the embodiment of the positioning mechanism of the eye protection apparatus of FIGS. 8A-8D, showing an attachment portion, a securing apparatus, a movement apparatus that comprises a guide rail, with ends of the guide rail capped to prevent the securing apparatus from falling out.

FIG. 9B is a front view of the movement apparatus of the positioning mechanism of the eye protection apparatus shown in FIG. 8A-9A.

FIG. 9C is a side view of the movement apparatus of the positioning mechanism of the eye protection apparatus shown in FIGS. 8A-9B, showing the securing apparatus at a first end of the guide rail of the movement apparatus.

FIG. 9D is a side view of the movement apparatus of the positioning mechanism of the eye protection apparatus shown in FIGS. 8A-9C, showing the securing apparatus at a second end of the guide rail of the movement apparatus, showing the securing apparatus tilted at an angle.

FIG. 10A is a side angled view of an eyeshield and a positioning mechanism of an embodiment of the eye protection apparatus, with a spring shown in the guide rail of one of the movement apparatus for mechanical control of the positioning mechanism, and the first cap of the movement apparatus and the securing apparatus serving as a locking mechanism together.

FIG. 10B is a front view of the first cap of the movement apparatus and the securing apparatus of the positioning mechanism shown in FIG. 10A, showing how the two parts work together as the locking mechanism.

FIG. 10C is a top view of the first cap of the movement apparatus of the positioning mechanism shown in FIGS. 10A-10B, showing slots of the locking mechanism.

FIG. 10D is a close-up side angled perspective view of the first cap of the movement apparatus and the securing apparatus of the positioning mechanism shown in FIG. 10A-10C, showing how the two parts work together as the locking mechanism, with a portion of the first cap of the movement apparatus partially cut out.

FIG. 10E is a close-up side angled perspective view of the first cap of the movement apparatus and the securing apparatus of the positioning mechanism shown in FIGS. 10A-10D, showing how the two parts work together as the locking mechanism, with a portion of the securing apparatus and a portion of the first cap of the movement apparatus partially cut out.

FIG. 11 is an exploded side perspective view of a positioning mechanism of an embodiment of the eye protection apparatus with a spring shown for mechanical control of the positioning mechanism, and a second cap with magnets, a securing apparatus with magnets, and a movement apparatus with magnets forming a locking mechanism.

FIG. 12A is an upper perspective view of an embodiment of the securing apparatus showing a magnet and a slot for the magnet, to serve as a part of a locking mechanism.

FIG. 12B is a below perspective view of an embodiment of the second cap of the movement apparatus showing a magnet and a slot for the magnet, to serve as a part of a locking mechanism.

FIG. 13 is an embodiment of the positioning mechanism that allows the user to electromechanically control the movement of a securing apparatus and an eyeshield secured to the securing apparatus (eyeshield not shown) along the movement apparatus.

FIG. 14 is an embodiment of the securing apparatus having a tilting mechanism that assist in tiling to an angle at a second end of a guide rail of a movement apparatus.

### DETAILED DESCRIPTION

One of the problems with headgears with attached eyeshields is that they are made with the intention of protecting the entire face and head of a user from hazards such as flying debris and chemical spills, and impacts in cases of accidents and falls, and are bulky and cumbersome for everyday use and activities, such as walking around the town on a sunny day. As an option, one can wear an eyeshield and a headgear, such as sunglasses and a cap, together at the same time to avoid wearing headgears with attached eyeshields. However, wearing a headgear and an eyeshield together can become cumbersome and inconvenient, as parts of the headgear can put pressure on the eyeshield, and the eyeshield consequently will transfer the pressure onto the wearer's face, such as the nose bridge, and head, such as the temple, causing discomfort.

For example, when a wearer wears a cap and eyeglasses together, the weight of the cap visor sits on top of the eyeglasses, creating discomfort on the nose bridge area of the wearer. The pressure from the cap's sweatband and the eyeglasses temples can also pose discomfort to the ear area of the wearer. Thus, wearers will often stow their eyeshield on top of their headgear when the eyeshield, such as sunglasses, is not in use to prevent additional pressure on their face or head. However, such a method of alleviating pressure is not secure and the eyeshield can fall or be knocked off of the headgear, causing damage or loss of the eyeshield.

Therefore, an eye protection apparatus that combines an eyeshield with a headgear while providing a mechanism to control the position of the eyeshield and alleviates pressure of the eyeshield on a user's face and head is needed.

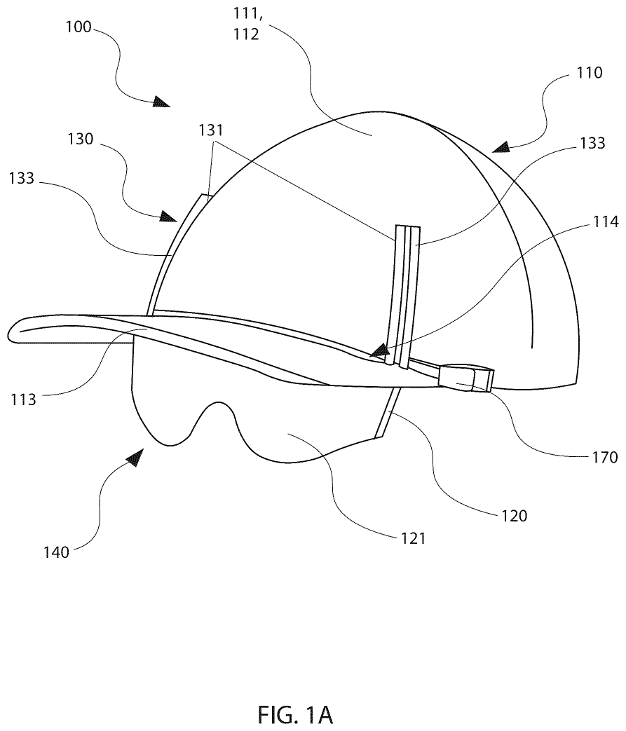

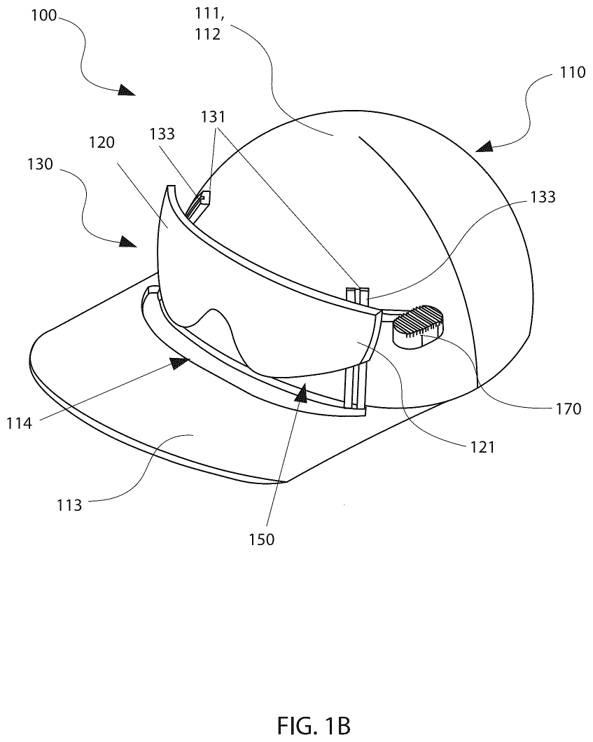

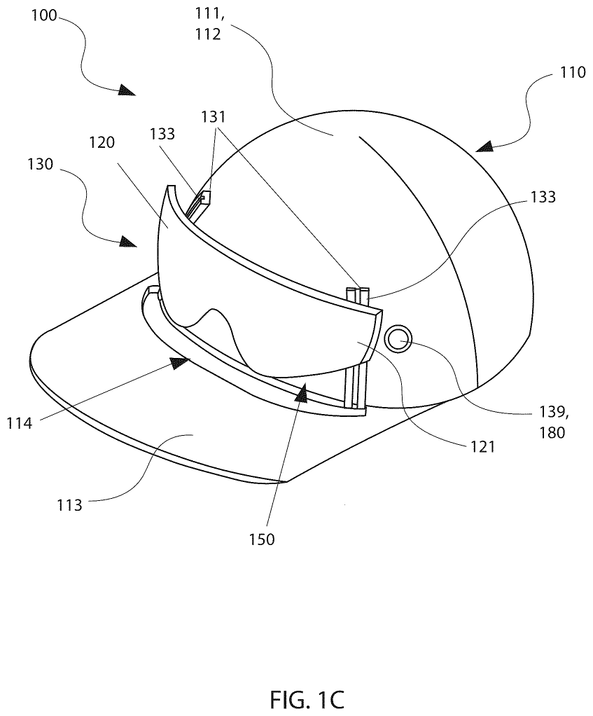

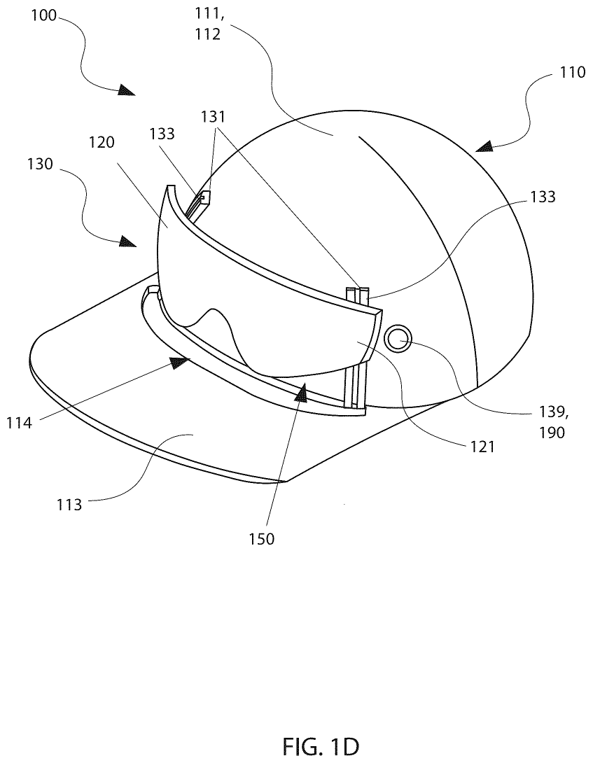

In general reference to FIGS. 1A-14, different embodiments of the eye protection apparatus 100, 200, 300, 400, 500, 600 are shown. The eye protection apparatus 100, 200, 300 comprises a headgear 110, 210, 310, an eye shield 120, 220, 320, and a positioning mechanism 130, 230, 330. In some embodiments, the eye protection apparatus 400, 500, 600 are shown without the headgear, comprising an eyeshield 420, 520 and a positioning mechanism 430, 530, 630, 830. The headgear 110, 210, 310 comprises a head covering 111, 211, 311 and a protruding portion 113, 213, 313 with a slit 114, 214, 314. The protruding portion 113, 213, 313 attaches to an outer portion 112, 212, 312 of the head covering 111, 211, 311. As shown in FIGS. 1A-7B, the headgear 110, 210, 310 can be a cap 110, 210, 310. However, other headgear, such as hats, cowboy hats, berets, helmets, and any other headgear known in the art can be a part of the eye protection apparatus. The protruding portion 113, 213, 313 can be a part of the headgear 110, 210, 310, such as a visor 113, 213, 313 of a cap 110, 210, 310 or can be a part of the eye protection apparatus that can be attached to an existing headgear. The headgear 110, 210, 310 can be comprised of one or more of fiber, fabric, metal polymer, and composite materials, or any other known materials in the art.

The eyeshield 120, 220, 320, 420, 520 has an eye-shielding surface 121, 221, 321, 421, 521. The eyeshield 120, 220, 320, 420, 520 can embody one or more properties, such as UV protection, radiation protection, visible light filtration, impact resistance, durability, polarization, optometric prescription, ANSI Z87.1, 3D binocular vision, multifocal, prism correction, high refraction index, scratch resistant, anti-reflective, water-repellant, light-responsive, and flash mirrored, and any other properties of eyeshield known in the art. At least one material for the eye-shielding surface 121, 221, 321, 421, 521 of the eyeshield 120, 220, 320, 420, 520 is glass, plastic, polycarbonate, trivex, high-index plastic, acetate, CR39 plastic, or polyacrylate, or any other material known in the art for making lenses for eyeshields. Eyeshield can be of any shape or function known in the art, such as an eye-visor, sunglasses, safety glasses, 3D-glasses, prescription glasses, or any other eyeshields known in the art, such as augmented reality glasses.

The positioning mechanism 130, 230, 330, 430, 530, 630, 830 comprises at least one attachment portion 131, 231, 331, 431, 531, 631, at least one securing apparatus 232, 332, 432, 532, 632 (not shown in FIGS. 1A-1D), 732, 832, 932 and at least one movement apparatus 133, 233, 333, 433, 533, 633, 833. The attachment portion 131, 231, 331, 431, 531, 631 attaches the positioning mechanism 130, 230, 330, 430, 530, 630, 830 to the head covering 111, 211, 311 of the headgear 110, 210, 310. The attachment portion 131, 231, 331, 431, 531, 631 can be attached to the headgear 110, 210, 310 by being sewn or glued onto the headgear 110, 210, 310, or by any other means of attachment known in the art, such as manufacturing directly onto the headgear 110, 210, 310. The attachment portion 131, 231, 331, 431, 531, 631 can be attached to an outer portion 112, 212, 312 of the head covering 111, 211, 311, an inner portion of the head covering 111, 211, 311, or through the head covering 111, 211, 311. The securing apparatus 132, 232, 332, 432, 532, 632, 732, 832, 932 secures the eyeshield 120, 220, 320, 420, 520 to the positioning mechanism 130, 230, 330, 430, 530, 630, 830. In FIGS. 1A-1D, the securing apparatus 132 is not visible due to the eyeshield 120 being positioned in front of it. The movement apparatus 133, 233, 333, 433, 533, 633, 833 is configured to allow movement of the securing apparatus 132, 232, 332, 432, 532, 632, 732, 832 and the eyeshield 120, 220, 320, 420, 520 secured to the securing apparatus 132, 232, 332, 432, 532, 632, 732, 832, 932 through the slit 114, 214, 314 of the protruding portion 113, 213, 313 of the headgear 110, 210, 310. The positioning mechanism can be comprised of one or more of fiber, fabric, metal polymer, and composite materials, and any other materials known in the art. The positioning mechanism 130, 230, 330, 430, 530, 630, 830 is configured to alleviate pressure of the eyeshield 120, 220, 320, 420, 520 on a user's face and head, since the eyeshield 120, 220, 320, 420, 520 is attached to the positioning mechanism 130, 230, 330, 430, 530, 630, 830 attached to the headgear 110, 210, 310 and therefore supported by the headgear 110, 210, 310.

The eyeshield 120, 220, 320, 420, 520 is retractable, as in it can be moved to one position from an original position and then counter moved to the original position. While the FIGS show the preferred embodiment of the eyeshield 120, 220, 320, 420, 520 pulled fully down or up, the eyeshield 120, 220, 320, 420, 520 can be in any position between fully down and fully up positions. Further, the movement can be sideways and is not limited to up and down vertical movement. While the FIGS show linear movement of the eyeshield 120, 220, 320, 420, 520, the movement of the eyeshield 120, 220, 320, 420, 520 is not limited to linear movement.

FIG. 1A shows an embodiment of the eye protection apparatus 100, with the positioning mechanism 130 comprising two attachment portions 131 attached to the outer portion 112 of the head covering 111 of the headgear 110, two movement apparatuses 133, and two securing apparatuses (not shown, placed behind the eyeshield surface 121). The eyeshield 120 with the eye-shielding surface 121 is held by the two securing apparatuses (not shown). The securing apparatuses are connected to a handle 170 for manual control 170 of the movement and position of the eyeshield 120. A user can also push or pull on the eyeshield 120 to manually control the eyeshield 120. In FIG. 1A, the eyeshield 120 is shown in the down position 140, fully pulled down through the slit 114 on the protruding portion 113 of the headgear 110.

FIG. 1B shows the embodiment of the eye protection apparatus 100 shown in FIG. 1A, with the eyeshield 120 shown in the up position 150, fully pulled up through the slit 114 on the protruding portion 113 of the headgear 110.

FIG. 1C shows the embodiment of the eye protection apparatus 100 shown in FIGS. 1A-1B, with the eyeshield 120 shown in the up position 150, fully pulled up through the slit 114 on the protruding portion 113 of the headgear 110. Instead of the handle 170 seen in FIGS. 1A-1B, a button 139 is provided for mechanically controlling 180 the movement.

FIG. 1D shows the embodiment of the eye protection apparatus 100 shown in FIGS. 1A-1C, with the eyeshield 120 shown in the up position 150, fully pulled up through the slit 114 on the protruding portion 113 of the headgear 110. The button 139 here is provided for electromechanically controlling 190 the movement. Actuator tab, button, or other methods known in the art can be used to control the movement. Automatic movements can be achieved using detents, springs, or magnetic materials.

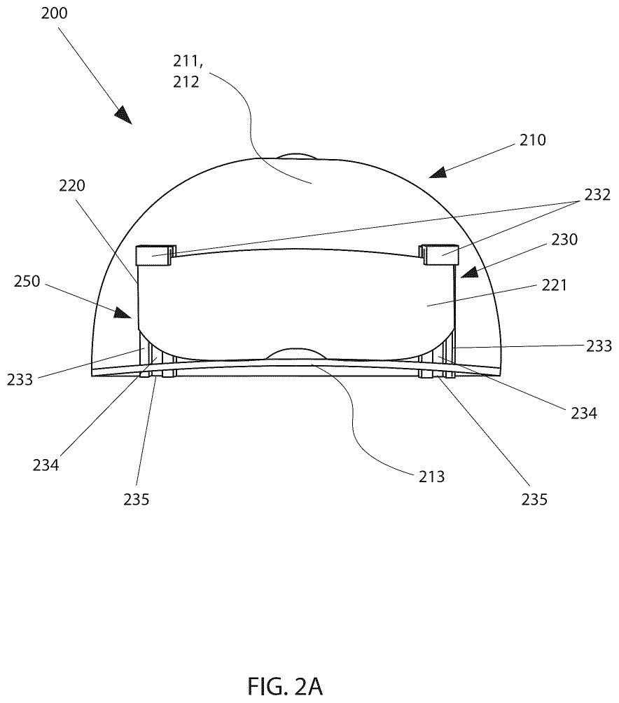

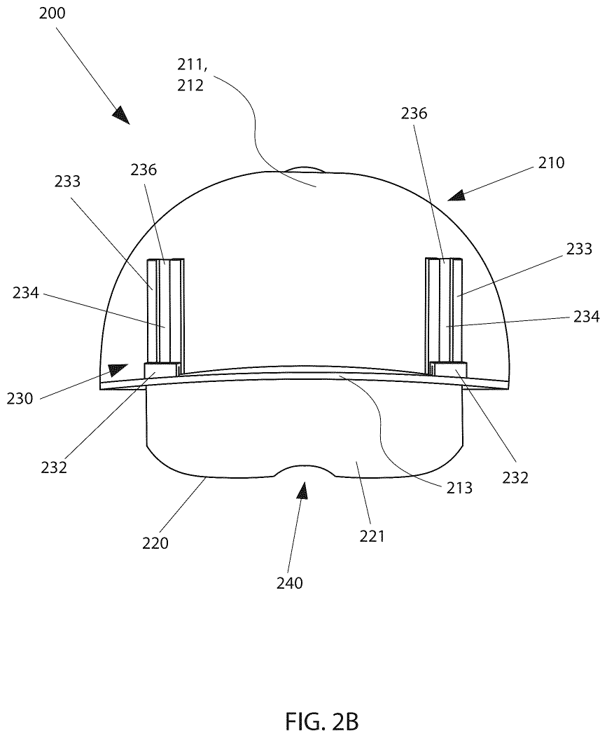

FIGS. 2A-2B show front views of another embodiment of the eye protection apparatus 200, with the positioning mechanism 230 comprising two attachment portions (not shown in FIGS. 2A-2B) attached to the outer portion 212 of the head covering 211 of the headgear 210, two securing apparatuses 232, and two movement apparatuses 233. In this embodiment, each movement apparatus 233 has a guide rail 234 for a securing apparatus 232 to move up and down. The guide rail 234 has a first end 235 (shown in FIG. 2A) at the bottom of the guide rail 234, and a second end 236 (shown in FIG. 2B) at the top of the guide rail 234. In FIG. 2A, the eyeshield 220 with eyeshield surface 221 is shown in the up position 250, fully pulled up through the slit 214 (not shown in FIGS. 2A-2b) on the protruding portion 213 of the headgear 210. In FIG. 2B, the eyeshield 220 with eyeshield surface 221 is shown in the down position 240, fully pulled down through the slit 214 (not shown in FIGS. 2A-2b) on the protruding portion 213 of the headgear 210.

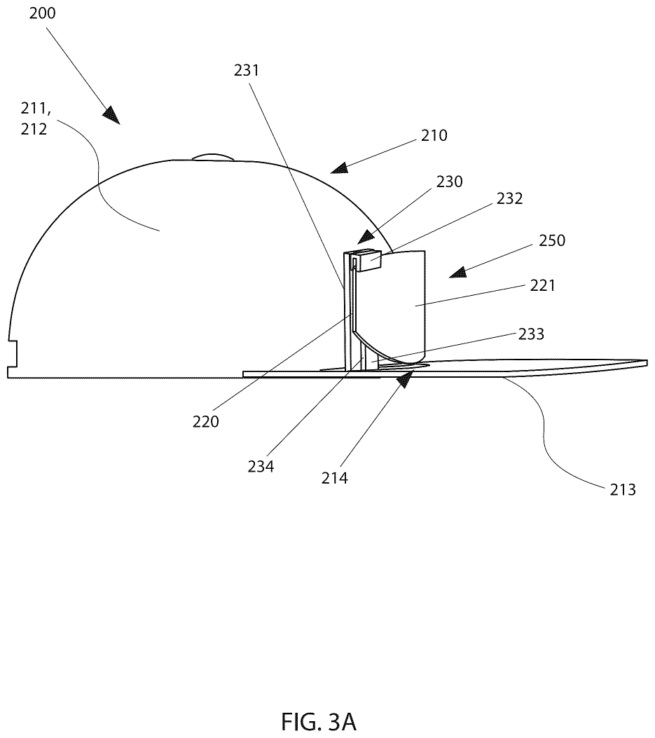

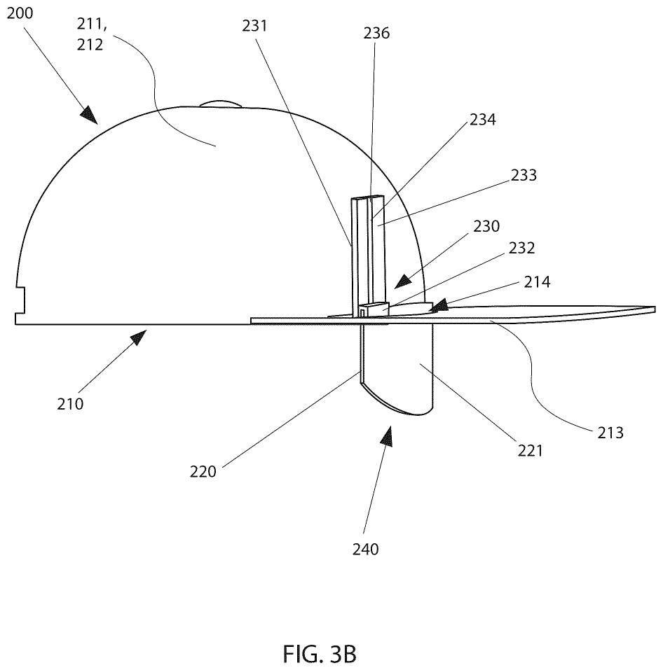

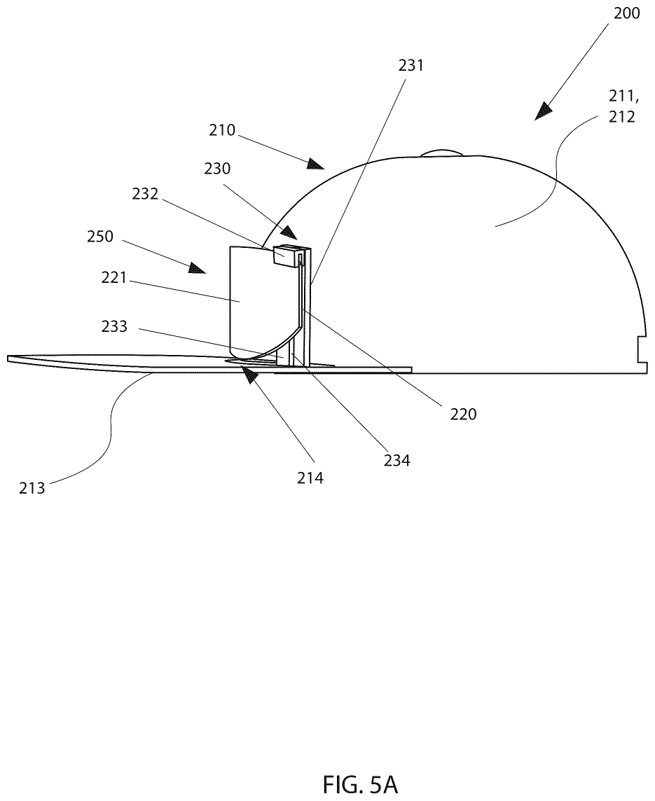

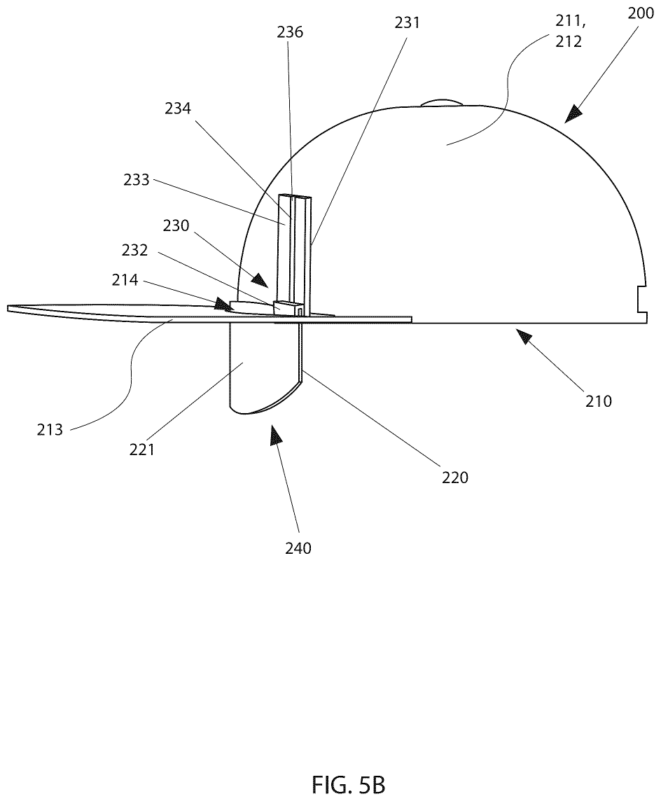

FIGS. 3A-3B show side views of the embodiment of the eye protection apparatus 200 in FIGS. 2A-2B, with one of the attachment portion 231 of the positioning mechanism 230 visible. FIGS. 5A-5B show opposite side views of the embodiment of the eye protection apparatus 200 in FIGS. 3A-3B. In FIGS. 3A and 5A, the eyeshield 220 with eyeshield surface 221 is shown in the up position 250, fully pulled up through the slit 214, visible in FIGS. 3A-3B and 5A-5B, on the protruding portion 213 of the headgear 210. In FIG. 3B and 5B, the eyeshield 220 with eyeshield surface 221 is shown in the down position 240, fully pulled down through the slit 214 on the protruding portion 213 of the headgear 210. In FIGS. 3A-3B and 5A-5B, you can also clearly see that the securing apparatus 232 is shaped to clamp the eyeshield 220 to secure it. Any other attachment means known in the art, such as glue, screws, tongue in groove, and other means can be used to attach the eyeshield 220 to the securing apparatus 232. The securing apparatus 232 slides up and down in the guide rail 234 of the movement apparatus 233.

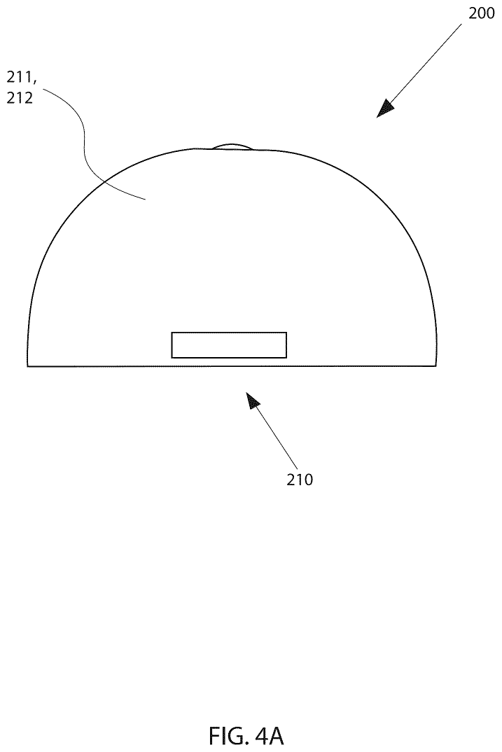

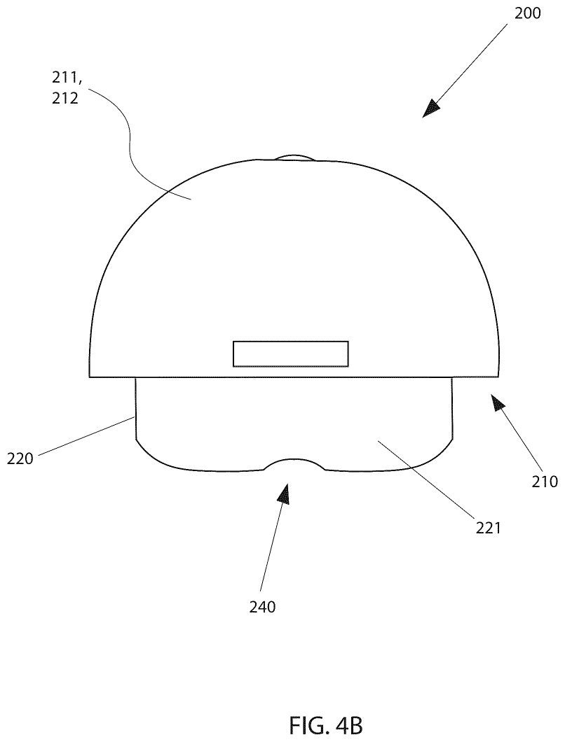

FIGS. 4A-4B show back views of the embodiment of the eye protection apparatus 200 in FIGS. 2A-3B, 5A-5B. In FIG. 4A, the eyeshield 220 (shown in FIG. 4B) is not visible because the eyeshield 220 is in fully up position. In FIG. 4B, the eyeshield 220 is shown in full down 240 position.

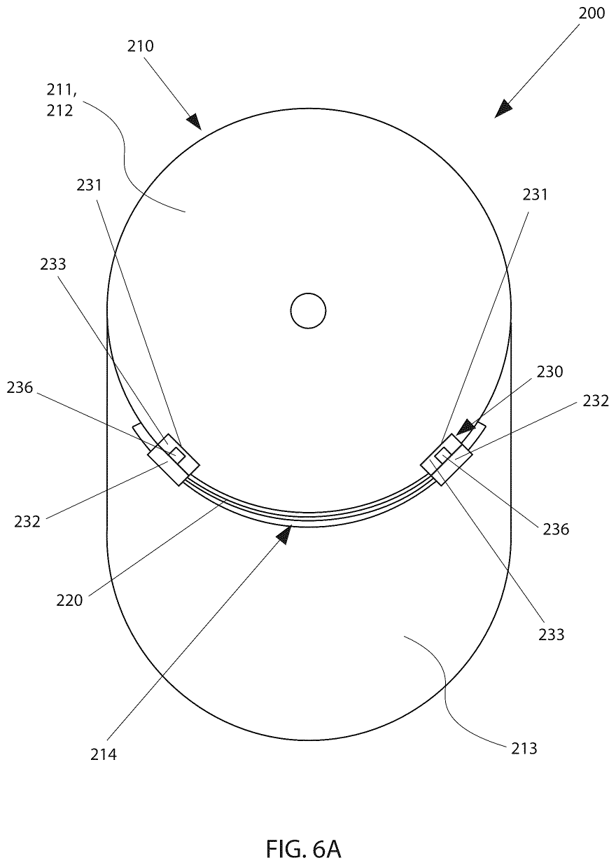

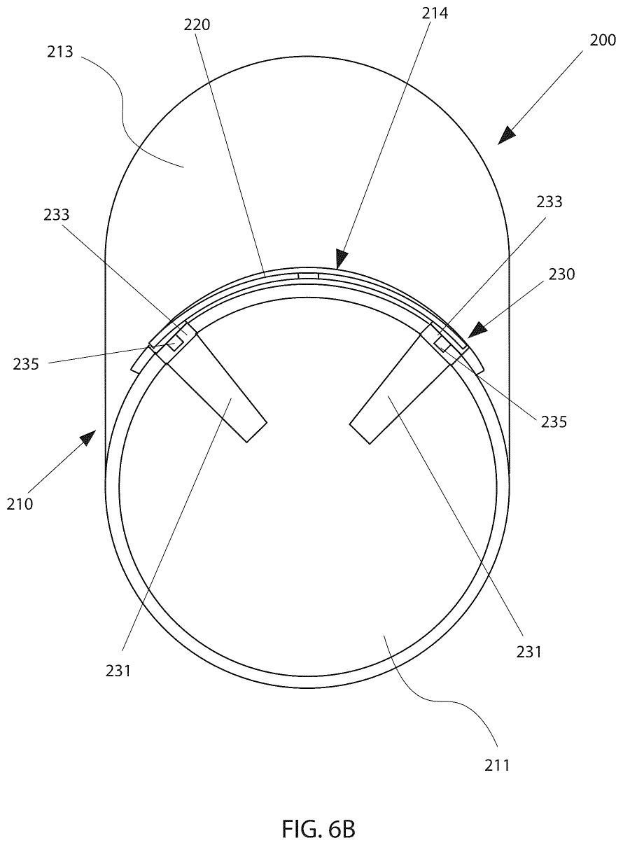

FIG. 6A shows a top view of the embodiment of the eye protection apparatus 200 in FIGS. 2A-5B, showing the securing apparatuses 232 of the positioning mechanism 230 in the guide rails 234 (not shown) of the movement apparatuses 233. The second ends 236 of the guide rails 234/the movement apparatuses 233 are shown. The slit 214 on the protruding portion 213 of the headgear 210 can be seen as having a curvature when viewed from the top or the bottom (shown in FIG. 6B), and the eyeshield 220 is shaped to fit through the slit 214. The eyeshield 220 and the slit 214 are not limited to the shapes seen in the FIGS. and can be of any shape or form known in the art as long as the eyeshield 220 is capable of fitting through the slit 214.

FIG. 6B shows a bottom view of the embodiment of the eye protection apparatus 200 in FIGS. 2A-6A, with the attachment portions 231 of the positioning mechanism 230 shown inside of the headgear 210, manufactured to be a part of the headgear 210. The attachment portions 231 can be attached the outer portion 212 (shown in FIG. 6A) of the head covering 211 of the headgear 210 instead. The first ends 235 of the guide rails 234 (not shown)/the movement apparatuses 233 are also shown.

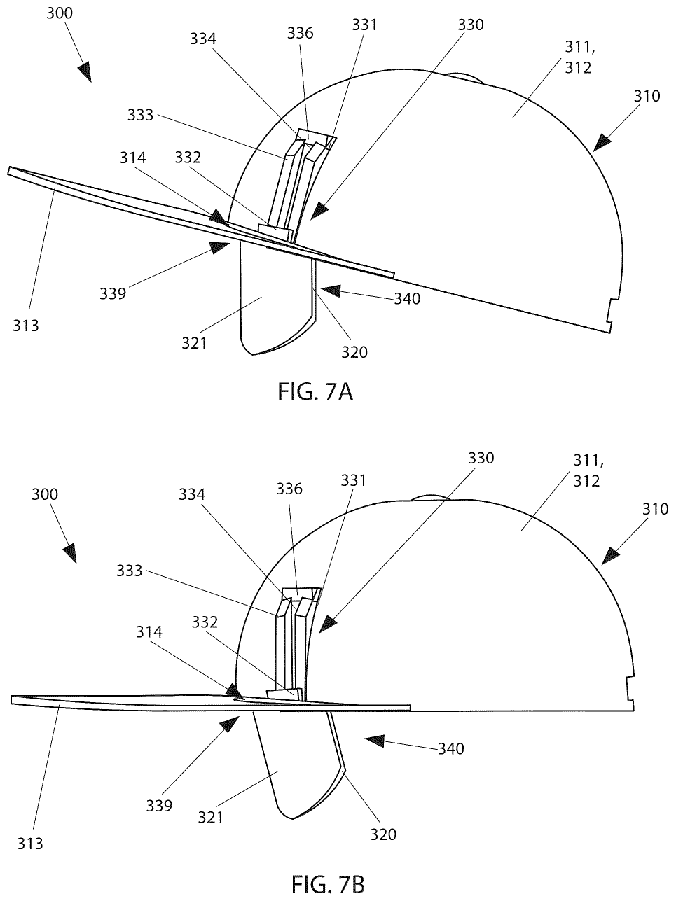

FIGS. 7A-7B show side views of another embodiment of the eye protection apparatus 300, having a headgear 310 comprising head covering 311 with an outer portion 312 and a protruding portion 313 with a slit 314, an eyeshield 320 with an eye-shielding surface 321, and a positioning mechanism 330. The positioning mechanism 330 comprises two attachment portions 331 (only one shown) attached to the outer portion 312 of the head covering 311 of the headgear 310, two securing apparatuses 332 (only one shown), and two movement apparatuses 333 (only one shown). In this embodiment, each movement apparatus 333 also has a guide rail 334 with a first end 335 (not shown) at the bottom and a second end 336 at the top for the securing apparatus 332 to move up and down. When the securing apparatus 332 and the attached eyeshield 320 are pulled all the way down to a down position 340, the movement apparatus 233 is shaped to allow the securing apparatus 332 and the attached eyeshield 320 to tilt an angle 339 at the first end 335 (not shown), allowing the eyeshield 320 to fit comfortably around the face for the user.

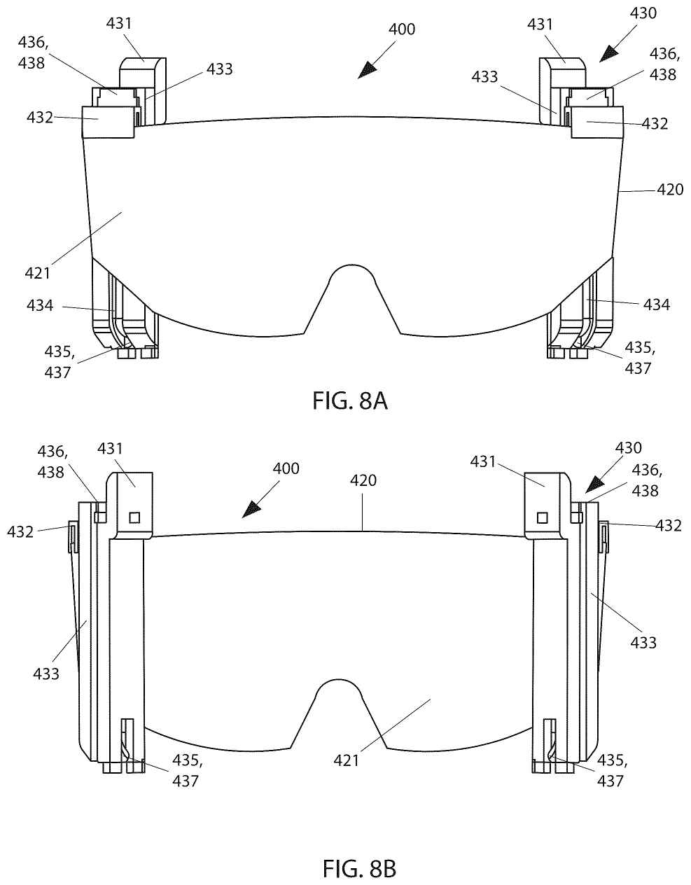

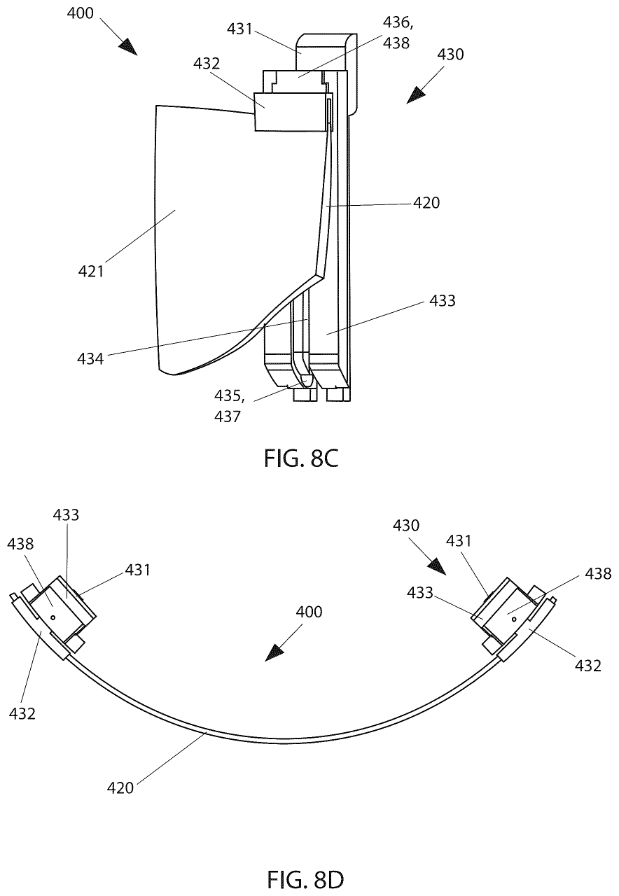

FIGS. 8A-8D show another embodiment of the eye protection apparatus 400, without a headgear. However, the eye protection apparatus 400 can be manufactured or attached onto a headgear and a headgear can be a component of the eye protection apparatus 400 as seen in FIGS. 1A-7B. The eye protection apparatus 400 comprises an eyeshield 420 with an eyeshield surface 421, and a positioning mechanism 430 having two attachment portions 431, two securing apparatuses 432, and two movement apparatuses 433. The movement apparatus 433 is formed to have a guide rail 434 (or a channel) having a first end 435 at the bottom and a second end 436 at the top. However, the movement apparatus 433 is not limited to be guide rails, and can be cam grooves, strings, or hinge points that allow movement of the securing apparatus 432. The movement apparatus 433 is shaped (curved) at the first end 435 to allow the securing apparatus 432 to tilt as shown in FIGS. 7A-7B. In the embodiment shown, a first cap 437 is attached to the first end 435 of the guide rail 434/movement apparatus 433 and prevents the securing apparatus 432 from falling out. A second cap 438 is attached to the second end 436 of the guide rail 434/movement apparatus 433 and prevents the securing apparatus 432 from falling out.

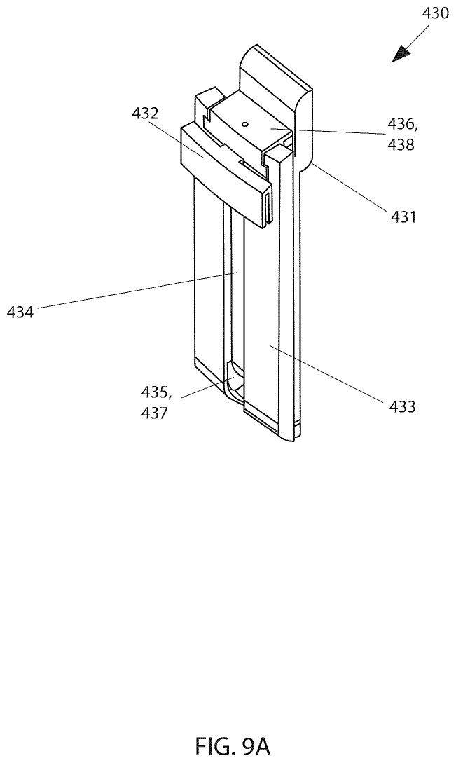

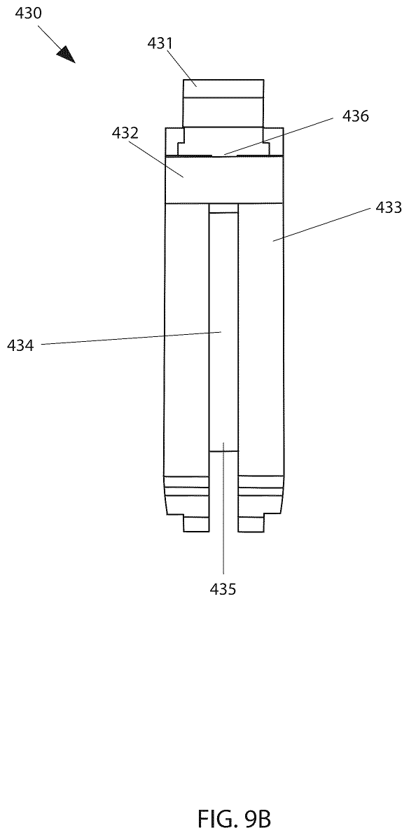

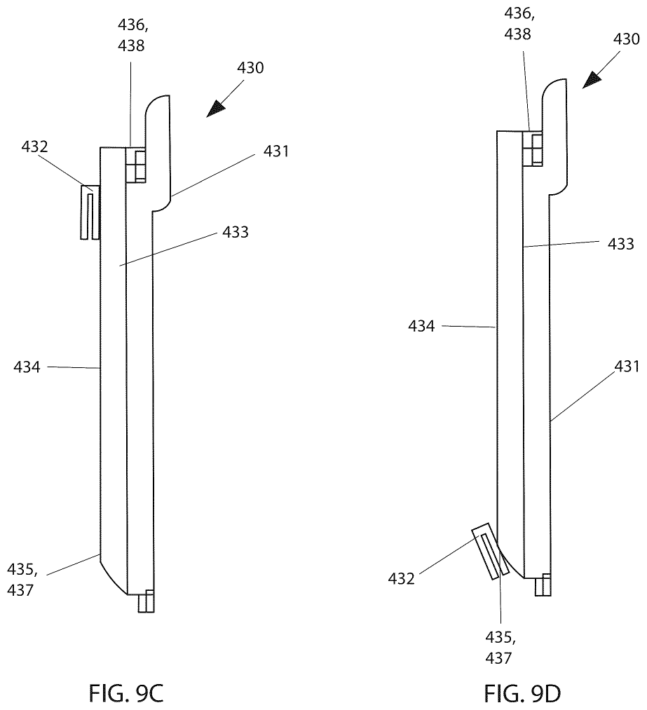

FIGS. 9A-9D show the positioning mechanism 430 of the eye protection apparatus 400 shown in FIGS. 8A-8D. FIG. 9A is a perspective view for providing a better view of the first cap 437 and the second cap 438. The first and second caps 437, 438 limit the movement of the securing apparatus 432 to within the guide rail 434 of the movement apparatus 433, thereby preventing the securing apparatus 432 from falling out or flying off the guide rail 434. FIG. 9B is a front view of the positioning mechanism 430, with the securing apparatus 432 within the guide rail 434 of the movement apparatus 433, but the first and second caps 437, 438 removed from the first and second ends 435, 436. FIG. 9C shows a side view of the positioning mechanism 430, showing how the securing apparatus 432 can be at the second end 436 (the top) of the guide rail 434 of the movement apparatus 433 and is prevented by the second cap 438 from falling off the top. FIG. 9D shows a side view of the positioning mechanism 430, showing how the securing apparatus 432 can be at the first end 435 (the bottom) of the guide rail 434 of the movement apparatus 433 and is prevented by the first cap 437 from falling out of the bottom. The tilt and angling of the securing apparatus 432 at the bottom of the guide rail 434 is also shown.

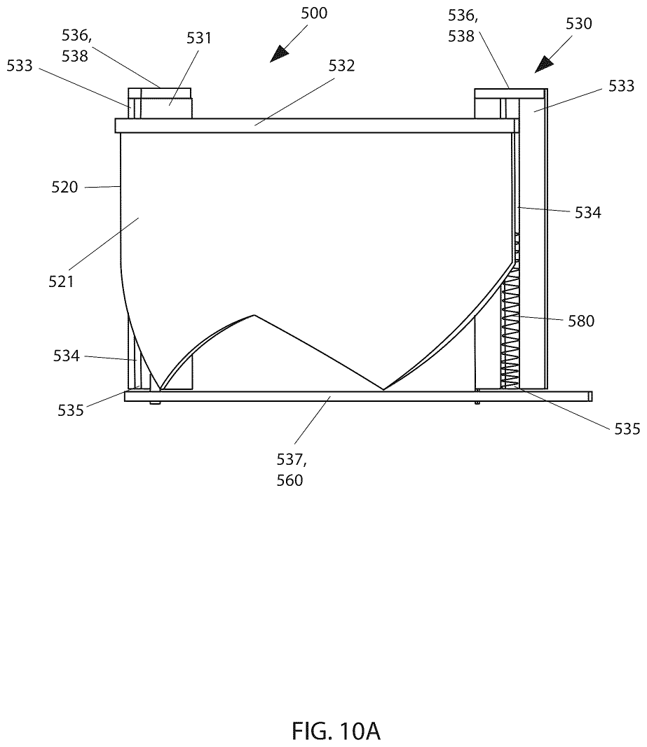

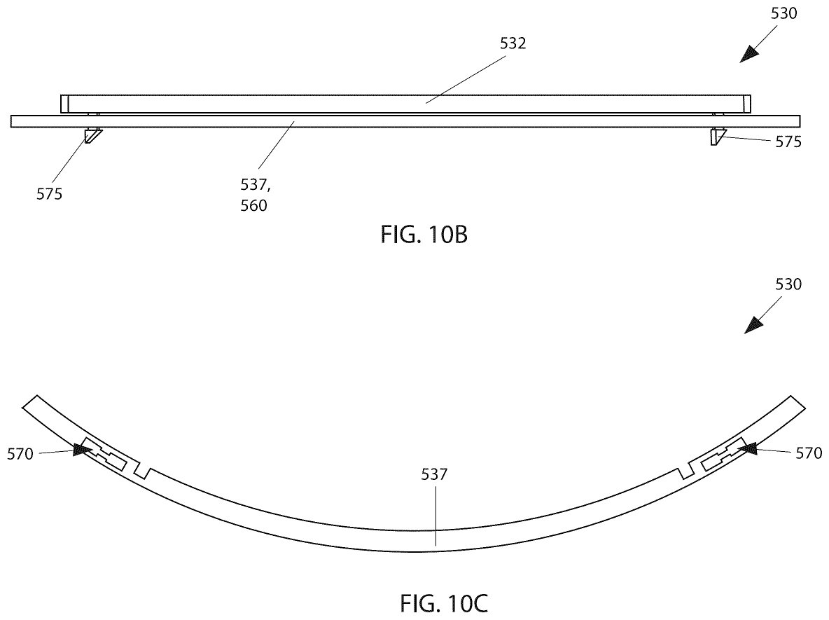

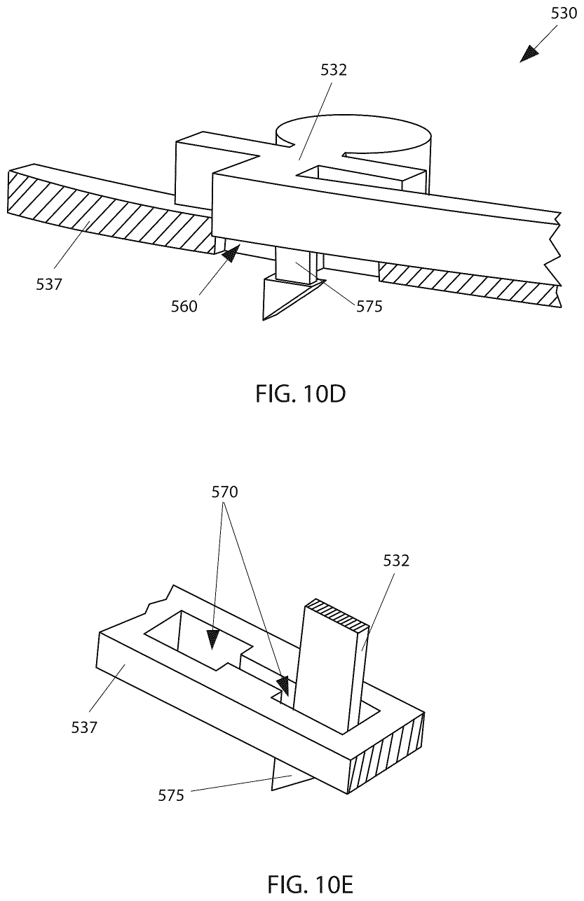

FIG. 10A shows another embodiment of the eye protection apparatus 500, without a headgear. The eye protection apparatus 500 comprises an eyeshield 520 with an eyeshield surface 521, and a positioning mechanism 530 having two attachment portions 531 (only one shown), one securing apparatus 532, and two movement apparatuses 533. The movement apparatus 533 is formed to have a guide rail 534 having a first end 535 at the bottom and a second end 536 at the top. The movement apparatus 533 has a first cap 537 attached to the first ends 535 of the guide rails 534/movement apparatuses 533 that prevents the securing apparatus 532 from falling out. Two second caps 538 are attached to the second ends 536 of the guide rails 534/movement apparatuses 533 and prevents the securing apparatus 532 from falling out. A locking mechanism 560 is provided for this embodiment (see FIGS. 10B-10E). Further, a spring 580 is placed inside each of the guide rail 534 of the movement apparatus 533 as a means to mechanically control 580 the movement, where the force from the compressed spring 580 will mechanically move the securing apparatus 532 and the eyeshield 520 attached back to the up position when unlocked.

FIGS. 10B-10E show the locking mechanism 560 in detail. FIG. 10B is a front view of the securing apparatus 532 locked with the first cap 537 of the movement apparatus 533, to prevent movement of the securing apparatus 532 and the attached eyeshield 520 (not shown) when locked, to hold the eyeshield 520 in the down position. In given embodiment, the first cap 537 has slots 560 for protruding portions 560 of the securing apparatus 532 to slide in and lock in place with. The slot 560 can be seen clearly in FIG. 10C and 10E. The shape of the slot is not limited to what is shown in FIGS. 10B-10E, and can be of any shape known in the art that works with the shape of the protruding portion 560 of the securing apparatus 532. The protruding portion 560 of the securing apparatus 532 can be also of any shape known in the art. The locking mechanism 560, is not limited to just being a hook 560 and slot 560 type as shown in IFGS. 10B-10E, and can be any locking mechanism known in the art.

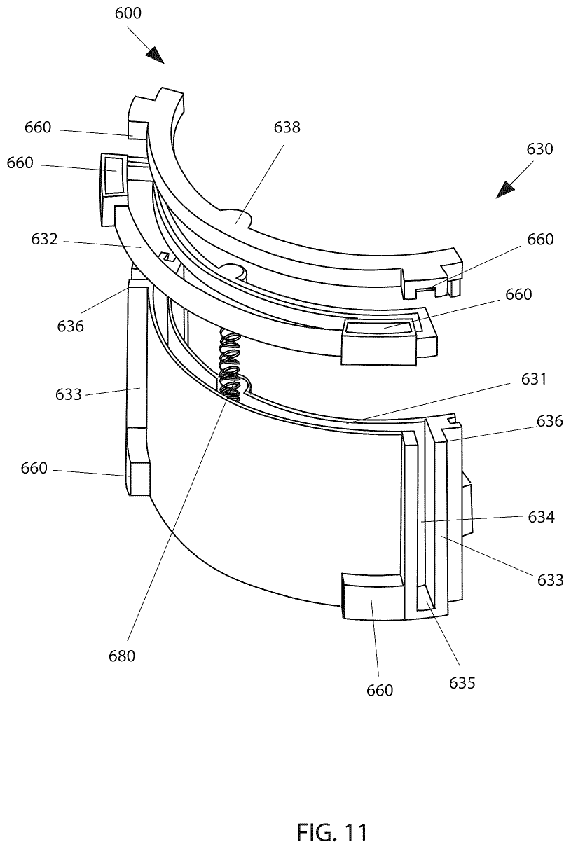

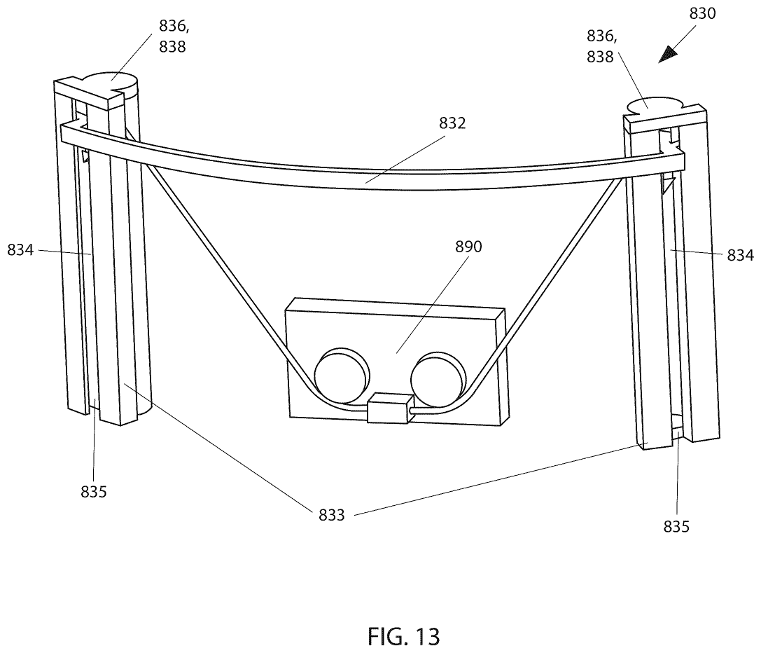

FIG. 11 shows an exploded side perspective view of yet another embodiment of the eye protection apparatus 600, without a headgear and an eyeshield. The eye protection apparatus 600 comprises a positioning mechanism 630 having one attachment portion 631, one securing apparatus 632 with two magnets 660, and two movement apparatuses 633 with magnets 660. Each of the movement apparatuses 633 is formed to have a guide rail 634 (only one shown) having a first end 635 at the bottom and a second end 636 at the top. The movement apparatus 633 is shaped so there is no need for a first cap at the first end 635. A second cap 638 with magnets 660 attach to the second ends 636 of the guide rails 634/movement apparatuses 633 and prevents the securing apparatus 632 from falling out. The magnets 660 in the second cap 638, the movement apparatuses 633, and the securing apparatus 632 work together as the locking mechanism 660. When the securing apparatus 632 (and the attached eyeshield) is at fully up position, the magnets 660 in the securing apparatus 632 and the magnets 660 in the second cap 638 lock the securing apparatus 632 in place, until the force between the magnet is overcome manually, mechanically, or electromagnetically. When the securing apparatus 632 (and the attached eyeshield) is at fully down position, the magnets 660 in the securing apparatus 632 and the magnets 660 in the movement apparatuses 633 lock the securing apparatus 632 in place, until the force between the magnet is overcome manually, mechanically, or electromagnetically. The force between the magnets 660 can be overcome manually by pushing or pulling on the securing apparatus 632 (or the eyeshield) or through mechanical or electromechanical means known in the art (see FIG. 13). Further, a spring 680 is placed inside the positioning mechanism 630 as a means to mechanically control 680 the movement. The force from the compressed spring 680 will mechanically move the securing apparatus 632 and the eyeshield attached back to the up position when unlocked from the down position. Other means of mechanically or electromechanically controlling the movement of the securing apparatus 632 known in the art can be used.

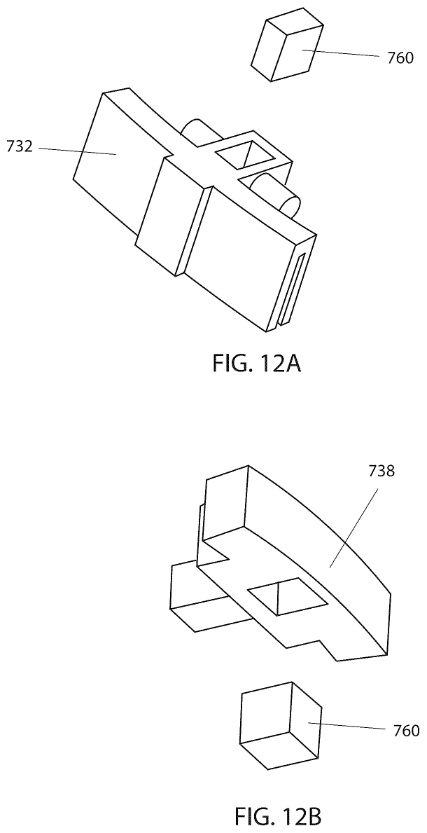

FIG. 12A shows an embodiment of the securing apparatus 732 with a slot for a magnet 760 for a locking mechanism 760. FIG. 12B shows an embodiment of the second cap 738 with a slot for a magnet 760 for the locking mechanism 760 in FIG. 12A.

FIG. 13 shows an embodiment of the positioning mechanism 830 that has one securing apparatus 832 and two movement apparatuses 833 each with a guide rail 834. In this embodiment, a motor 890 with gear wheels housed inside that wind and rewind wire or string is shown. The motor 890 allows the user to electromechanically control 890 the movement of the securing apparatus 832 and the eyeshield (not shown) secured to the securing apparatus 832 (eyeshield not shown) along the movement apparatuses 833. Other means of electromechanically controlling 890 the movement of the securing apparatus 832 also can be used. The motor 890 can be coupled to a button (as seen in FIGS. 1C-1D) or any other means of controlling the motor.

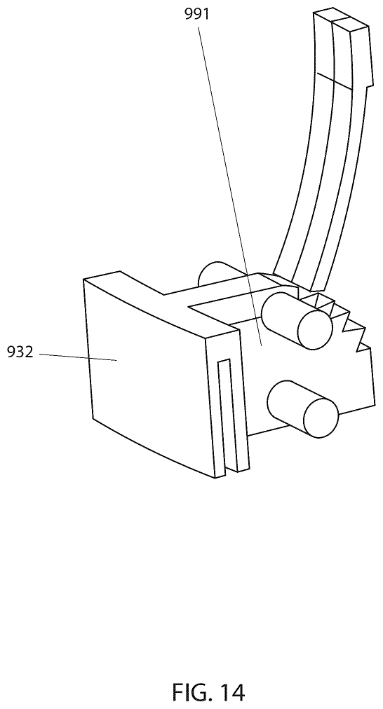

FIG. 14 is an embodiment of the securing apparatus 932 having a tilting mechanism 991 that assists in tiling to an angle (see FIGS. 7A and 7B) at a second end of the guide rail of the movement apparatus (not shown). Other tiling mechanisms known in the art can be also used instead of the embodiment shown.

The indefinite articles “a” and “an,” as used herein in the specification and in the claims, unless clearly indicated to the contrary, should be understood to mean “at least one.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to.

A preferred embodiment of the present disclosure has been described with reference to the preferred embodiments illustrated in the attached drawing figures. However, equivalents may be employed and substitutions made herein without departing from the scope of the invention as recited in the claims. Therefore, it will be apparent to those skilled in the art that many changes and modifications may be made without departing from the claimed subject matter in its broader aspects. The appended claims are intended to cover all such changes and modifications as fall within the true spirit and scope of the invention.

What is claimed is:

1. An eye protection apparatus comprising:
a headgear comprising:
a head covering; and
a protruding portion with a slit, attached to an outer portion of the head covering;
an eyeshield having an eye-shielding surface; and
a positioning mechanism comprising:
at least one attachment portion for attaching the positioning mechanism to the head covering of the headgear;
at least one securing apparatus that secures the eyeshield to the positioning mechanism; and
at least one movement apparatus configured to allow movement of the at least one securing apparatus and the eyeshield secured to the at least one securing apparatus through the slit of the protruding portion of the headgear;
wherein the positioning mechanism is configured to alleviate pressure of the eyeshield on a user's face and head.

2. The eye protection apparatus of claim 1, wherein the headgear is a cap and the protruding portion is a visor of the cap.

3. The eye protection apparatus of claim 1, wherein the at least one movement apparatus is comprised of a guide rail and the at least one securing apparatus is configured to move along the guide rail of the at least one movement apparatus.

4. The eye protection apparatus of claim 1, wherein the positioning mechanism is configured to allow a user to manually control the movement of the at least one securing apparatus and the eyeshield secured to the at least one securing apparatus through the slit of the protruding portion of the headgear.

5. The eye protection apparatus of claim 1, wherein the positioning mechanism is configured to allow a user to mechanically control the movement of the at least one securing apparatus and the eyeshield secured to the at least one securing apparatus through the slit of the protruding portion of the headgear.

6. The eye protection apparatus of claim 1, wherein the positioning mechanism is configured to allow a user to electromechanically control the movement of the at least one securing apparatus and the eyeshield secured to the at least one securing apparatus through the slit of the protruding portion of the headgear.

7. The eye protection apparatus of claim 6, wherein the positioning mechanism further comprises a button configured to allow the user to electromechanically control the movement of the at least one securing apparatus and the eyeshield secured to the at least one securing apparatus through the slit of the protruding portion of the headgear.

8. The eye protection apparatus of claim 1, wherein the eyeshield embodies one or more properties, the one or more properties comprising: UV protection, radiation protection, visible light filtration, impact resistance, durability, polarization, optometric prescription, ANSI Z87.1, 3D binocular vision, multifocal, prism correction, high refraction index, scratch resistant, anti-reflective, water-repellant, light-responsive, and flash mirrored.

9. The eye protection apparatus of claim 8, wherein at least one material for the eye-shielding surface of the eyeshield is glass, plastic, polycarbonate, trivex, high-index plastic, acetate, CR39 plastic, or polyacrylate.

10. The eye protection apparatus of claim 1, wherein the headgear is comprised of one or more of fiber, fabric, metal polymer, and composite materials, and wherein the positioning mechanism is comprised of one or more of fiber, fabric, metal polymer, and composite materials.

11. The eye protection apparatus of claim 1, wherein the at least one movement apparatus is further configured to allow the at least one securing apparatus and the eyeshield secured to the at least one securing apparatus to tilt at an angle when the eyeshield is positioned below the slit.

12. The eye protection apparatus of claim 1, wherein a position of the eyeshield is locked by a locking mechanism of the positioning mechanism.

13. An eye protection apparatus comprising:
a cap comprising:
a head covering; and
a visor with a slit, attached to an outer portion of the head covering;
an eyeshield having an eye-shielding surface; and
a positioning mechanism configured to alleviate pressure of the eyeshield on a user's face and head comprising:
at least one attachment portion for attaching the positioning mechanism to the head covering of the cap;
at least one securing apparatus that secures the eyeshield to the positioning mechanism; and
at least one guide rail configured to:
allow movement of the at least one securing apparatus and the eyeshield secured to the at least one securing apparatus along the guide rail and through the slit of the visor of the cap; and
allow the at least one securing apparatus and the eyeshield secured to the at least one securing apparatus to tilt at an angle when the eyeshield is positioned below the slit.

14. The eye protection apparatus of claim 13, wherein the positioning mechanism is configured to allow a user to manually control the movement of the at least one securing apparatus and the eyeshield secured to the at least one securing apparatus along the guide rail and through the slit of the visor of the cap.

15. The eye protection apparatus of claim 13, wherein the positioning mechanism is configured to allow a user to mechanically control the movement of the at least one securing apparatus and the eyeshield secured to the at least one securing apparatus along the guide rail and through the slit of the visor of the cap.

16. The eye protection apparatus of claim 13, wherein the positioning mechanism is configured to allow a user to electromechanically control the movement of the at least one securing apparatus and the eyeshield secured to the at least one securing apparatus along the guide rail and through the slit of the visor of the cap.

17. The eye protection apparatus of claim 16, wherein the positioning mechanism further comprises a button configured to allow the user to electromechanically control the movement of the at least one securing apparatus and the eyeshield secured to the at least one securing apparatus along the guide rail and through the slit of the visor of the cap.

18. The eye protection apparatus of claim 13, wherein the eyeshield embodies one or more properties, the one or more properties comprising: UV protection, radiation protection, visible light filtration, impact resistance, durability, polarization, optometric prescription, ANSI Z87.1, 3D binocular vision, multifocal, prism correction, high refraction index, scratch resistant, anti-reflective, water-repellant, light-responsive, and flash mirrored; and wherein at least one material for the eye-shielding surface of the eyeshield is glass, plastic, polycarbonate, trivex, high-index plastic, acetate, CR39 plastic, or polyacrylate.

19. The eye protection apparatus of claim 13, wherein a position of the eyeshield is locked by a locking mechanism of the positioning mechanism.

20. An eye protection apparatus for attachment to a headgear, comprising:
an eyeshield having an eye-shielding surface; and
a positioning mechanism configured to alleviate pressure of the eyeshield on a user's face and head comprising:
at least one attachment portion for attaching the positioning mechanism to a head covering of the headgear;
at least one securing apparatus that secures the eyeshield to the positioning mechanism; and
at least one movement apparatus configured to:
allow movement of the at least one securing apparatus and the eyeshield secured to the at least one securing apparatus through a slit in a protruding portion of the headgear; and
allow the at least one securing apparatus and the eyeshield secured to the at least one securing apparatus to tilt at an angle when the eyeshield is positioned below the slit in the protruding portion of the headgear.
